(19) United States
(12) Patent Application Publication
(10) Pub. No.: US 2023/0075093 A1
(43) Pub. Date: Mar. 9, 2023

(54) TEXTILE-TYPE DRY ELECTRODE PLATE

(71) Applicant: Hui-Ling CHANG, New Taipei City (TW)

(72) Inventors: Chien-Hung LAI, New Taipei City (TW); Chang-Sheng WU, New Taipei City (TW); Ke-Chi FU, Taichung City (TW); Chung-Chuan CHANG, Kinmen County (TW)

(21) Appl. No.: 17/923,218

(22) PCT Filed: Jun. 9, 2020
(86) PCT No.: PCT/CN2020/095131

§ 371 Date: Nov. 3, 2022

(51) Int. Cl. A61B 5/05; A61N 1/04

(52) U.S. Cl. CPC A61B 5/05; A61N 1/04; A61B 5/257

(57) ABSTRACT

The present disclosure discloses a textile-type multi-channel high-sensitivity sensing dry electrode plate including a body, a conductive cloth and at least one electrode connection part. The body is a sheet-shaped body. The conductive cloth is arranged on one side of the body. The conductive cloth includes at least one electrode part. The electrode connection part is arranged on the body, through the body and electrically connected to the electrode part of the conductive cloth. Moreover, the electrode connection part transmits currents onto the conductive cloth, so that the electrode part of the conductive cloth generates an effect of an electrotherapy or a thermotherapy, or both the electrotherapy and the thermotherapy simultaneously. The electrode part receives physiological electric signals transmitted by a human body and transmits the physiological electric signals back to an apparatus through the electrode connection part to perform a physiological measurement.

### CROSS-REFERENCE TO RELATED APPLICATION

This application is the National Phase of PCT International Application No. PCT/CN2020/095131 filed on Jun. 9, 2020. The entire disclosures of the above application are all incorporated herein by reference.

### BACKGROUND OF THE DISCLOSURE

### Technical Field

The present disclosure relates to an electrotherapy apparatus area, and especially relates to a textile-type multi-channel high-sensitivity sensing dry electrode plate which is used to contact the human body to receive or transmit physiological electric signals.

### Description of Related Art

It is known that currently the common sport injuries, degeneration or other problems, with the use of 3C electronic products leading to many bad postures, result in physical pain, such as soreness, pain, numbness and other symptoms due to prolonged bowing and hunchback causing muscle imbalance. Patients will firstly use patches, anti-inflammatory pain relievers or massages to relieve pain once these symptoms occur. However, these methods may not completely cure these symptoms, so patients will seek professional treatment from a physical therapist, or under the evaluation of the physical therapist, patients can purchase physical therapy instruments or apparatuses to use electrotherapy or thermotherapy by themselves.

These apparatuses for electrotherapy or thermotherapy have a patch and a low frequency therapy apparatus. After the low frequency therapy apparatus is electrically connected to the patch, the current generated by the low frequency therapy apparatus is transmitted to the patch, so that the electrode part on the patch can perform physical therapy on the sore place of the patient. In the past, the patches used for electric signal transmission all have a water-rich layer (gel, hydrogel) for signal transmission; the resistance is not easy to control due to the capacitive effect of the water-rich layer; the main reasons for poor control include the following:

1. Hydrogel is not easy to store, and will dry out and cause inconsistent conductivity; the difference between batches is also not easy to control.

2. The amount of gel applied manually each time is different, resulting in a difference in impedance, which affects the measurement. Due to the above reasons, the signal will be saturated or distorted, and the subsequent software determination may have errors or incorrect determination.

3. Some high frequency electrical stimulators or electric signal transmitters will have a capacitive effect if they are used with the water-rich glue layer, causing delay and distortion, which will make the therapeutic effect ineffective.

### SUMMARY OF THE DISCLOSURE

The main object of the present disclosure is to solve the conventional deficiencies. In order to achieve the object mentioned above, the present disclosure provides a textile-type multi-channel high-sensitivity sensing dry electrode plate which includes a body, a conductive cloth and at least one electrode connection part. The body is a sheet-shaped body. The conductive cloth is arranged on one side of the body. The conductive cloth includes at least one electrode part. The electrode connection part is arranged on the body, through the body and electrically connected to the electrode part of the conductive cloth. Moreover, the electrode connection part transmits signals onto the conductive cloth, so that the electrode part of the conductive cloth generates an effect of (namely, performs) an electrotherapy or a thermotherapy, or both the electrotherapy and the thermotherapy simultaneously; meanwhile the electrode part receives physiological electric signals transmitted by a human body and transmits the physiological electric signals back to an apparatus through the electrode connection part.

In an embodiment of the present disclosure, the textile-type multi-channel high-sensitivity sensing dry electrode plate further includes an adhesive layer arranged on the one side of the body. The adhesive layer and the conductive cloth are arranged on the same one side of the body.

In an embodiment of the present disclosure, the body is an adhesive bandage.

In an embodiment of the present disclosure, the body is non-adhesive.

In an embodiment of the present disclosure, the body is a bandage.

In an embodiment of the present disclosure, the conductive cloth is a silver fiber cloth, a fabrication of a nanometer silver wire or a printing of a conductive paste.

In an embodiment of the present disclosure, the conductive paste is a thermal-transfer-type conductive gel.

In an embodiment of the present disclosure, the electrode part of the conductive cloth is a hollow-shaped structure with dot-shaped pores, strip-shaped pores or mesh-shaped pores.

In an embodiment of the present disclosure, the conductive cloth includes two of the electrode parts symmetrically arranged on the one side of the body.

In an embodiment of the present disclosure, the electrode connection part is arranged through the body and electrically connected to the two of the electrode parts respectively.

In an embodiment of the present disclosure, the textile-type multi-channel high-sensitivity sensing dry electrode plate further includes a wire electrically connected between the electrode part of the conductive cloth and the electrode connection part for a signal transmission.

Besides, the present disclosure uses silver fiber cloth, nanometer silver wire or conductive paste (thermal-transfer-type conductive gel); a conductive cloth made of these conductive materials is cut after an elastic back glue is applied to the conductive cloth; with various hot pressing or sewing methods, the conductive cloth is fixed on an electrode plate; the traction of the conductive cloth is used as a wire or a specific totem of the conductive cloth is used as an electrode, in order to improve the electric signal transmission carrier (the conductive cloth); the water-rich wet electrode plate (patch) is replaced by a dry electrode. The present disclosure is used in electrocardiograph signal industry, electromyography signal rehabilitation industry and other industries, to achieve the beneficial effect of improving the measurement accuracy. The present disclosure is used in electrical stimulation related industries; the transmission of electric signals to the human body can be more accurate, so that the electrical stimulation system can transmit more accurate signals to the epidermis, muscles and the human body; especially, the muscle stimulation (EMS/ES) system can more accurately receive signals which are obtained by measuring functional movement of the muscles, and can apply electrical stimulation with corresponding parameters to achieve the effect of rehabilitation. In the high frequency electrical stimulation system, the present disclosure can effectively transmit signals and energy, reduce distortion and achieve better therapeutic effects.

### BRIEF DESCRIPTION OF THE DRAWINGS

FIG. 1 shows a three-dimensional schematic diagram of the appearance of the first embodiment of the electrode plate of the present disclosure.

FIG. 2 shows a schematic diagram of the outer surface of FIG. 1.

FIG. 3 shows a schematic diagram of the state in which the electrode plate of FIG. 1 is used together with the low frequency electrotherapy apparatus.

FIG. 4 shows a three-dimensional schematic diagram of the appearance of the knee pad of the second embodiment of the electrode plate of the present disclosure.

FIG. 5 shows a schematic diagram of the other side of the electrode plate of FIG. 4.

FIG. 6 shows a schematic diagram of another embodiment of the conductive cloth of the electrode plate of the present disclosure.

FIG. 7 shows a schematic diagram of yet another embodiment of the conductive cloth of the electrode plate of the present disclosure.

FIG. 8 shows a schematic diagram of still another embodiment of the conductive cloth of the electrode plate of the present disclosure.

### DETAILED DESCRIPTION

Regarding the technical contents and detailed descriptions of the present disclosure, it is now described with the drawings as follows:

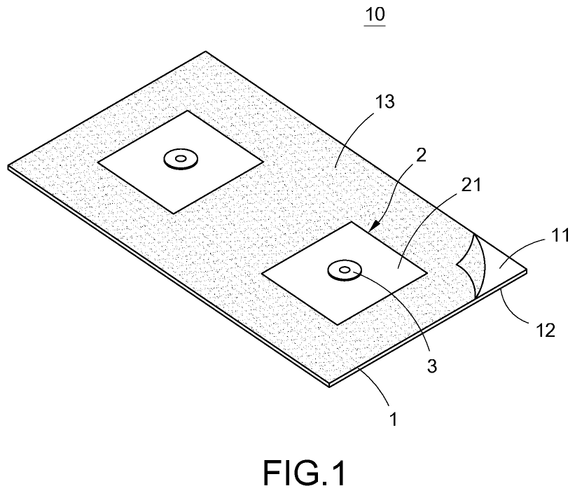

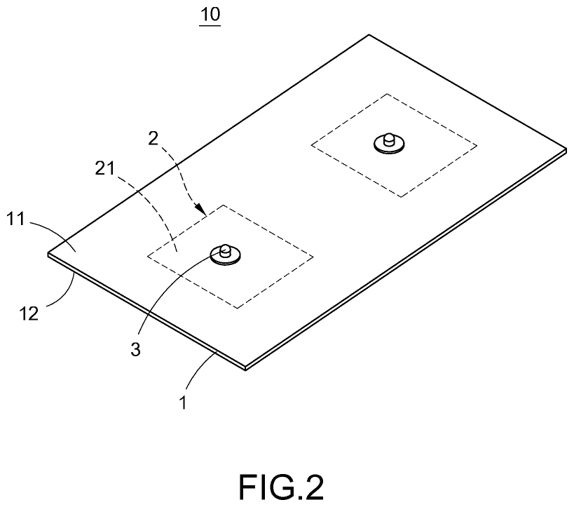

FIG. 1 shows a three-dimensional schematic diagram of the appearance of the first embodiment of the electrode plate of the present disclosure. FIG. 2 shows a schematic diagram of the outer surface of FIG. 1. As shown in FIG. 1 and FIG. 2, the electrode plate 10 (namely, the textile-type multi-channel high-sensitivity sensing dry electrode plate 10) of the present disclosure includes a body 1, a conductive cloth 2 and at least one electrode connection part 3. After the electrode plate 10 is electrically connected to an external low frequency electrotherapy apparatus (not shown in FIG. 1 or FIG. 2), the electrotherapy or the thermotherapy can be performed, or both the electrotherapy and the thermotherapy can be performed simultaneously; meanwhile, the electrode part 21 receives physiological electric signals transmitted by a human body and transmits the physiological electric signals back to a device of the low frequency electrotherapy apparatus through the electrode connection part 3.

The body 1 is a sheet-shaped body, and includes an inner surface 11 and an outer surface 12. The electrode plate 10 further includes an adhesive layer 13 arranged on the inner surface 11. The adhesive layer 13 is an adhesive, so that the body 1 can be adhered to the surface of the patient's skin. In the embodiment, the body 1 may have the adhesive layer 13 with adhesion such as the adhesive bandage material which is the stretchable tape type, while the patch material of the adhesive bandage is a nonwoven cloth with good air permeability, stretchability and flexibility. Besides, the body 1 may be a non-adhesive bandage.

The conductive cloth 2 is a silver fiber cloth, which is elastic and has a certain tensile strength. The interwoven silver fiber cloth has more strength and is not easy to break and lose the conductive function. During production, the silver fiber cloth is cut after an elastic back glue is applied to the silver fiber cloth, and is fixed on the inner surface 11 of the body 1 by various hot pressing or sewing methods, and is arranged on the same side as the adhesive layer 13. The conductive cloth 2 at least includes an electrode part 21. The usage/presence/traction of the conductive cloth 2 is used as a wire or a specific pattern of the conductive cloth 2 is used as the electrode part 21, to achieve the effect of receiving and transmitting signals. The conductive cloth 2 has extremely low resistance, uniform and stable resistivity, and fiber features. The conductive cloth 2 further has some advantages; first, the conductive cloth 2 is not easy to cause non-uniform resistance or disconnection due to factors such as pulling, and is a good signal transmission carrier; second, transmitting signals by the conductive cloth 2 does not require to be through the water-rich material, such as the hydrogel, while the purpose of the signal transmission can be achieved by using the dry conductive cloth 2, thereby eliminating the problem of non-uniform resistance in the man-made water-rich gel process. In the embodiment, in addition to the silver fiber cloth, the conductive cloth 2 may also be a fabrication of a nanometer silver wire or a printed matter formed by printing the conductive paste. In some embodiments, the conductive paste is a thermal-transfer-type conductive gel.

The electrode connection part 3 is arranged on the body 1, through the body 1 and electrically connected to the electrode part 21 of the conductive cloth 2. After the electrode connection part 3 is electrically connected to the low frequency electrotherapy apparatus (not shown in FIG. 1 or FIG. 2), with the output current, the conductive cloth 2 has the effect of the electrotherapy or the thermotherapy. In the embodiment, the electrode connection part 3 is a male connector of a button.

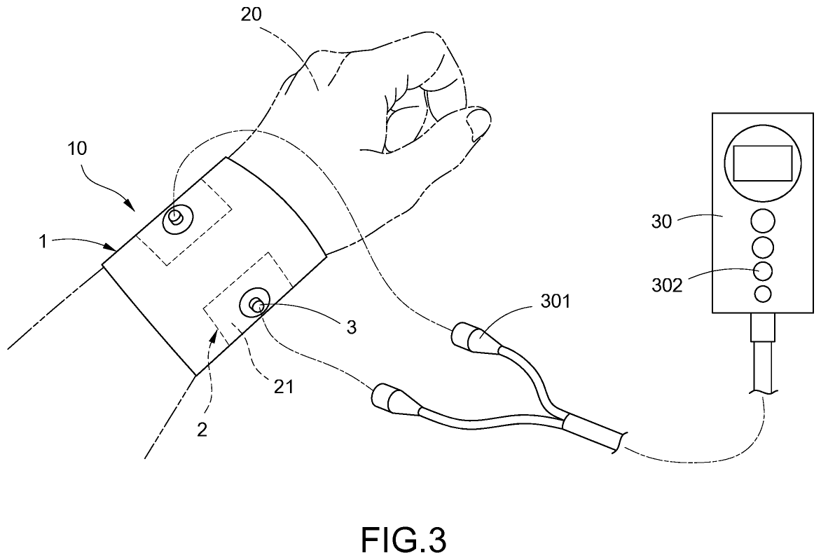

FIG. 3 shows a schematic diagram of the state in which the electrode plate of FIG. 1 is used together with the low frequency electrotherapy apparatus. As shown in FIG. 3, when the electrode plate 10 of the present disclosure is used, the adhesive layer 13 on the body 1 is pasted on the surface of the patient's skin 20.

The electrode plate 10 is pasted on the surface of the patient's skin 20, so that the electrode part 21 contacts the patient's skin 20; meanwhile the electrode connection part 3 is exposed on the outer surface 12 of the body 1. An electrode connector 301 of the low frequency electrotherapy apparatus 30 is electrically connected onto the electrode connection part 3. After a button 302 of the low frequency electrotherapy apparatus 30 is pressed, the current outputted by the low frequency electrotherapy apparatus 30 is transmitted onto the electrode connection part 3 through the electrode connector 301, and then the current is transmitted onto the conductive cloth 2 by the electrode connection part 3, so that the electrode part 21 of the conductive cloth 2 can perform the treatment course of the electrotherapy or the thermotherapy, or both the electrotherapy and the thermotherapy simultaneously; meanwhile the electrode part 21 receives physiological electric signals transmitted by the patient's body and transmits the physiological electric signals back to a device of the low frequency electrotherapy apparatus 30 through the electrode connection part 3.

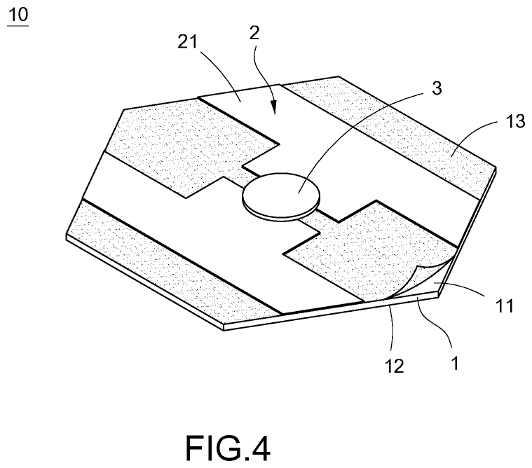

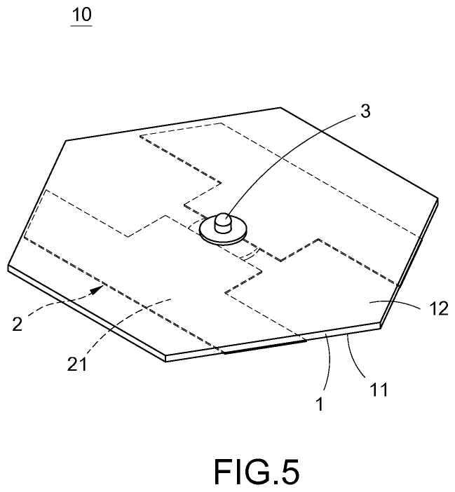

FIG. 4 shows a three-dimensional schematic diagram of the appearance of the second embodiment of the electrode plate of the present disclosure. FIG. 5 shows a schematic diagram of the outer surface of FIG. 4. As shown in FIG. 4 and FIG. 5, this embodiment is substantially the same as the first embodiment, except that the conductive cloth 2 on the body 1 of the electrode plate 10 includes two electrode parts 21 which are symmetrical and convex-shaped. After the electrode connection part 3 is arranged through the body 1, the electrode connection part 3 is electrically connected to the two electrode parts 21 respectively.

As shown in FIG. 3, the electrode plate 10 is pasted on the surface of the patient's skin 20, so that the electrode part 21 contacts the patient's skin 20; meanwhile the electrode connection part 3 is exposed on the other side of the body 1. An electrode connector 301 of the low frequency electrotherapy apparatus 30 is electrically connected onto the electrode connection part 3. After the button 302 of the low frequency electrotherapy apparatus 30 is pressed, the current outputted by the low frequency electrotherapy apparatus 30 is transmitted onto the electrode connection part 3 through the electrode connector 301, and then the current is transmitted onto the conductive cloth 2 by the electrode connection part 3, so that the electrode part 21 of the conductive cloth 2 can perform the treatment course of the electrotherapy or the thermotherapy, or both the electrotherapy and the thermotherapy simultaneously; meanwhile the electrode part 21 receives physiological electric signals transmitted by the patient's body and transmits the physiological electric signals back to a device of the low frequency electrotherapy apparatus 30 through the electrode connection part 3.

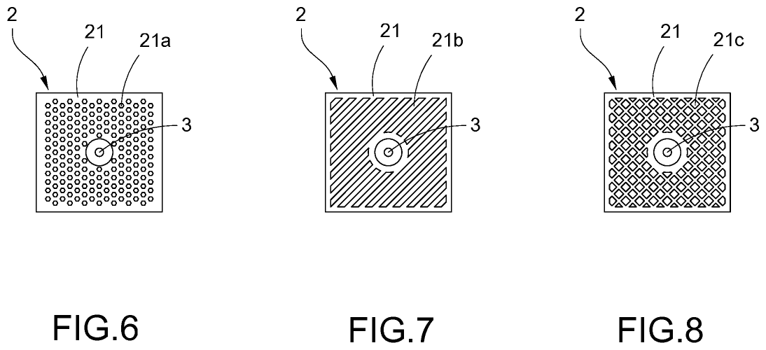

FIG. 6, FIG. 7 and FIG. 8 show schematic diagrams of various embodiments of the conductive cloth of the electrode plate of the present disclosure. As shown in FIG. 6, FIG. 7 and FIG. 8, the conductive cloth 2 of the present disclosure is made of a silver fiber cloth or nanometer silver wires, or by printing a conductive paste (such as a thermal-transfer-type conductive gel). During processing, the electrode part 21 of the conductive cloth 2 is designed to have various hollow-shaped structures such as dot-shaped pores 21a, strip-shaped pores 21b and mesh-shaped pores 21c; the electrode part 21 is fixed on one side of the body 1 of the electrode plate 10 by adhesion, thermal transfer, printing or other techniques.

Since the conductive cloth 2 is a hollow-shaped structure with dot-shaped pores 21a, strip-shaped pores 21b or mesh-shaped pores 21c, the body 1 (such as the bottom fabric) has high elasticity and better stretching performance. When the body 1 is stretched, the conductive cloth 2 will not be broken due to insufficient interlayer adhesion and tension, and will not lose conductive performance.

Moreover, the electrode plate 10 further includes a wire electrically connected between the electrode part 21 of the conductive cloth 2 and the electrode connection part 3 for signal transmission.

With the design of the structure of the electrode plate 10 of the present disclosure mentioned above, the electrode plate 10 may be used as an electric signal transmission carrier in medical equipment or sport equipment, such as related accessories for the electrotherapy, the thermotherapy and so on; or the electrode plate 10 may be used as a signal receiving carrier in electrocardiograph signal system, electromyography signal system or rehabilitation monitoring system, such as related protectors or clothing textiles; or the electrode plate 10 can be used as multi-channel transmission (receiving and outputting simultaneously) in functional sport clothing, muscle training clothing for electronic muscle stimulation (EMS) or heart rate monitoring clothing.

The descriptions mentioned above are only the embodiments of the present disclosure, and are not intended to limit the scope of the present disclosure. Therefore, any equivalent changes made by using the contents of the descriptions or drawings of the present disclosure are equally included in the scope of the present disclosure.

What is claimed is:

1. A textile-type dry electrode plate comprising:
a body being a sheet-shaped body;
a conductive cloth arranged on one side of the body and comprising at least one electrode part; and
at least one electrode connection part arranged on the body, through the body and electrically connected to the electrode part of the conductive cloth,
wherein the electrode connection part is configured to transmit a current onto the conductive cloth, so that the electrode part of the conductive cloth is configured to generate an effect of an electrotherapy or a thermotherapy, or both the electrotherapy and the thermotherapy simultaneously;
the electrode part is configured to receive physiological electric signals transmitted by a human body and transmit the physiological electric signals back to an apparatus through the electrode connection part to measure the physiological electric signals.

2. The textile-type dry electrode plate of claim 1, further comprising an adhesive layer arranged on the one side of the body, wherein the adhesive layer and the conductive cloth are arranged on the same one side of the body.

3. The textile-type dry electrode plate of claim 2, wherein the body is an adhesive bandage.

4. The textile-type dry electrode plate of claim 1, wherein the body is non-adhesive.

5. The textile-type dry electrode plate of claim 4, wherein the body is a bandage.

6. The textile-type dry electrode plate of claim 1, wherein the conductive cloth is a silver fiber cloth, a fabrication of nanometer silver wires or a printing of a conductive paste.

7. The textile-type dry electrode plate of claim 6, wherein the conductive paste is a thermal-transfer-type conductive gel.

8. The textile-type dry electrode plate of claim 1, wherein the electrode part of the conductive cloth is a hollow-shaped structure with dot-shaped pores, strip-shaped pores or mesh-shaped pores.

9. The textile-type dry electrode plate of claim 1, wherein the conductive cloth comprises two electrode parts, and the two electrode parts are symmetrically arranged on the one side of the body.

10. The textile-type dry electrode plate of claim 9, wherein the electrode connection part is arranged through the body and electrically connected to the two of the electrode parts respectively.

11. The textile-type dry electrode plate of claim 1, further comprising a wire electrically connected between the electrode part of the conductive cloth and the electrode connection part for a signal transmission.
